(19) United States
(12) Patent Application Publication
(10) Pub. No.: US 2020/0113789 A1
(43) Pub. Date: Apr. 16, 2020

(54) FEEDING TUBE ORIFICE DILATOR

(71) Applicant: Henry Brejt, Brooklyn, NY (US)

(72) Inventor: Henry Brejt, Brooklyn, NY (US)

(21) Appl. No.: 16/714,195

(22) Filed: Dec. 13, 2019

Related U.S. Application Data
(60) Provisional application No. 62/779,444, filed on Dec. 13, 2018.

(51) Int. Cl. A61J 15/00; A61M 29/00

(52) U.S. Cl. CPC A61J 15/0019; A61M 29/00; A61J 15/0026

(57) ABSTRACT

The present invention relates to a plurality of feeding tube orifice dilators (10, 20, 30, 40) that allows users to dilate the Percutaneous endoscopic gastrostomy (PEG) orifice comprising a proximal end portion (14) and a distal end portion (16) interconnected by an elongated body (12); a series of measuring marks (18) is applied to the dilators (10, 20, 30, 40) which are increased progressively in diameter through the proximal end portion (14) to the distal end portion (16); and a soft tipped wire (31) inserted into the proximal end portion (14) of dilator (30). Further, dilator (20) has small openings (14a) near the proximal end portion (14) and is hollow on the inside. Further, the dilators (10, 20, 30, 40) are inserted and removed prior to inserting the feeding tube after the opening is enlarged.

### FIELD OF THE INVENTION

The present invention relates to a plurality of feeding tube orifice (also known as “PEG tube stoma”) dilators for easily replacing feeding tubes into a percutaneous endoscopic gastrostomy (PEG) tube orifice. More particularly, the present invention relates to feeding tube orifice dilator that allows users to dilate the PEG orifice once it contracted to a diameter too small to manually reinstall the feeding tube.

### BACKGROUND AND PRIOR ART

Generally, dilation tools are used, inter alia, in endoscopic ENT surgery, but also in other specialist areas of medicine. They are used to remove pathological stenoses by virtue of an expandable section being introduced into the region of the stenosis and the section being dilated. A simple solution for dilation lies in use of a balloon catheter which is disadvantageous if the balloon bursts and “pumping work” is required by the operator if a gas is used. If use is made of an incompressible fluid, the biocompatibility thereof, in turn, needs to be taken into account. Purely mechanical dilation tools are also known; although these usually cannot be dilated in an ideal manner, they do permit sensitive force feedback with fewer hand movements. Mechanical dilation tools are often disadvantageous in that comparatively large gaps are formed between the spreading jaws in the spread state, which contributes to an increased risk of trauma due to a pressure load which is too high and due to clamping. As a result of this, overcoats were developed for mechanical dilation tools, which overcoats are intended to compensate these gaps, homogenize the introduction of force into the tissue and prevent the ingress of tissue into the dilation mechanism.

In recent years, there have been an increasing number of cases in which, after dilating a constricted portion by a catheter with an expandable body and avulsing the catheter with the expandable body from the constricted portion, the dilated state of the constricted portion is observed by use of an ultrasonic catheter or the like, and, when constriction remains, the catheter with the expandable body once avulsed is again inserted into the constricted portion to perform re-dilation.

Therefore, the inserting feeding tubes can be immensely difficult. The feeding tube can come out of the abdomen, and if not replaced immediately, the opening tends to shrink and close. Replacing the feeding tube can cause discomfort and pain of the patient. Even if the feeding tube is replaced, often times, the feeding tube is replaced with a tube with a smaller diameter.

There are few prior art documents related to the dilation tool with expandable covering tube. U.S. Pat. No. 10,045,762 B2, assigned to Karl Storz S E and Co K G. The prior art document relates to a dilation device including a dilation instrument which includes a distal radially expandable section which is coupled via an elongate shank to a handling section, and an expandable covering with a closed distal end and an open proximal end, for the expansion section of the dilation instrument. The covering has a shank section extending along the shank of the dilation instrument. The open proximal end of the covering is detachably coupled to the distal end of the handling section of the dilation instrument. Furthermore, the covering is disclosed per se, which consists of a mesh material and has a shank section, which is connected to a coupling sleeve at the open proximal end.

Another U.S. Pat. No. 8,845,726B2, assigned to VertiFlex Inc. The prior art document relates to a dilator that facilitates implantation of an interspinous spacer is provided. The dilator includes a proximal portion and a tapered distal portion interconnected by an elongated body portion. The tapered distal portion is ideally suited for splitting ligamentous tissue for creating a posterior midline pathway through the supraspinous ligament as well as for distracting the adjacent spinous processes. Two oppositely located and longitudinally extending channels or grooves are formed in the outer surface of the dilator for stabilizing the dilator with respect to the spinous processes. An accompanying cannula together with the dilator form a system for the distraction of the adjacent spinous processes, stabilization of the spinous processes with respect to the system and creation of a working channel for the implantation of an interspinous spacer.

Yet another US patent application number 20050216047A1, assigned to Terumo Corp. The document relates to a catheter includes an elastically radially expandable body encompassed by an elastically radially expandable slitted sleeve. Before the body is expanded, the slit is closed but is opened when the body and the sleeve are expanded. When the catheter is used to dilate a constricted portion of a blood vessel, the opened slit defines a recess into which the constricted portion projects, in order to resist slippage of the body off the constricted portion. When the body and the sleeve are allowed to contract, the sleeve forces the body into a generally cylindrical shape to facilitate subsequent re-dilation of the constricted portion.

In order to solve the above mentioned prior art problems, to provide the feeding tube orifice dilator that allows users to dilate the Percutaneous endoscopic gastrostomy (PEG) orifice once it contracted to a diameter too small to manually reinstall the feeding tube.

Also, the purpose of this device to provide a medical professional with safe, simple, and quick device that could be used in a doctor's office, nursing home or even patient's home without having to send the patient to the emergency room.

The information disclosed in this background of the disclosure section is only for enhancement of understanding of the general background of the invention and should not be taken as an acknowledgement or any form of suggestion that this information forms the prior art already known to a person skilled in the art.

### SUMMARY OF THE INVENTION

One or more drawbacks of conventional dilator and additional advantages are provided through the present invention relates to a plurality of feeding tube orifice dilators that allows users to dilate the Percutaneous endoscopic gastrostomy (PEG) orifice. Additional features and advantages are realized through the technicalities of the present disclosure. Other embodiments and aspects of the disclosure are described in detail herein and are considered to be a part of the claimed disclosure.

In an embodiment of the present invention relates to a plurality of feeding tube orifice dilators that allows users to dilate the Percutaneous endoscopic gastrostomy (PEG) orifice once it contracted to a diameter too small to manually reinstall the feeding tube. The plurality of feeding tube orifice dilators that allows users to dilate the PEG orifice comprises a proximal end portion and a distal end portion interconnected by an elongated body; a series of measuring marks is applied to the dilators which is increased progressively in diameter through the proximal end portion to the distal end portion; and a soft tipped wire inserted into the proximal end portion to the distal end portion.

It is to be understood that the aspects and embodiments of the disclosure described above may be used in any combination with each other. Several of the aspects and embodiments may be combined to form a further embodiment of the disclosure.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

It is to be noted, however, that the appended drawings illustrate only typical embodiments of the present subject matter and are therefore not to be considered for limiting its scope, for the invention may admit to other equally effective embodiments. The detailed description is written with reference to the accompanying figures. In the figures, the left-most digit(s) of a reference number identifies the figure in which the reference number first appears. The same numbers are used throughout the figures to reference the like features and components. Some embodiments of system or methods in accordance with embodiments of the present subject matter are now described, by way of example, and with reference to the accompanying figures, in which:

FIG. 1 illustrates schematic view of feeding tube orifice dilator (Model 1) with series of measuring marks, in accordance with an embodiment of the present subject matter.

FIG. 2 illustrates schematic view of feeding tube orifice dilator (Model 2) with series of measuring marks and is hollow on the inside, in accordance with an embodiment of the present subject matter.

FIG. 3 illustrates schematic view of feeding tube orifice dilator (Model 3) with soft tipped wire, in accordance with an embodiment of the present subject matter.

FIG. 4 illustrates schematic view of feeding tube orifice dilator (Model 4) with proximal and distal end portions, in accordance with an embodiment of the present subject matter.

The figures depict embodiments of the present subject matter for the purposes of illustration only. A person skilled in the art will easily recognize from the following description that alternative embodiments of the structures and methods illustrated herein may be employed without departing from the principles of the disclosure described herein.

### DETAILED DESCRIPTION OF THE INVENTION

While the embodiments of the disclosure are subject to various modifications and alternative forms, specific embodiments thereof have been shown by way of example in the figures and will be described below. It should be understood, however, that it is not intended to limit the disclosure to the particular forms disclosed, but on the contrary, the disclosure is to cover all modifications, equivalents, and alternatives falling within the scope of the disclosure.

The terms “comprises”, “comprising”, or any other variations thereof used in the disclosure, are intended to cover a non-exclusive inclusion, such that a device, system, assembly that comprises a list of components does not include only those components but may include other components not expressly listed or inherent to such system, or assembly, or device. In other words, one or more elements in a system or device proceeded by “comprises . . . a” does not, without more constraints, preclude the existence of other elements or additional elements in the system or device.

An embodiment of the present invention relates to the feeding tube orifice dilator which is a tool that allows users to dilate the percutaneous endoscopic gastrostomy (PEG) orifice once it contracted to a diameter too small to manually reinstall the feeding tube. The dilator can also be used to replace a feeding tube with a larger diameter feeding tube if needed.

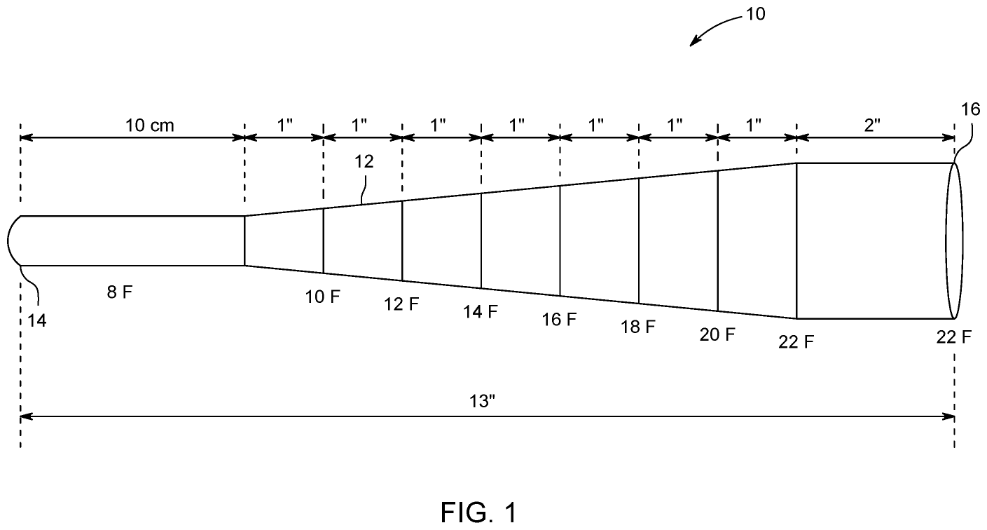

As shown in FIG. 1 illustrating schematic view of feeding tube orifice dilator with series of measuring marks, in accordance with an embodiment of the present subject matter. A plurality of feeding tube orifice dilators (10, 20, 30, 40) comprises one feeding tube orifice dilator 10 having a proximal end portion 14 and a distal end portion 16 interconnected by an elongated body 12. This feeding tube orifice dilator 10 starts out with size 8 F tip×10 cm made from flexible but firm plastic, rubber or silicone and then slowly increases to size at 22 F and then continues as 22 F, as shown in Model 1.

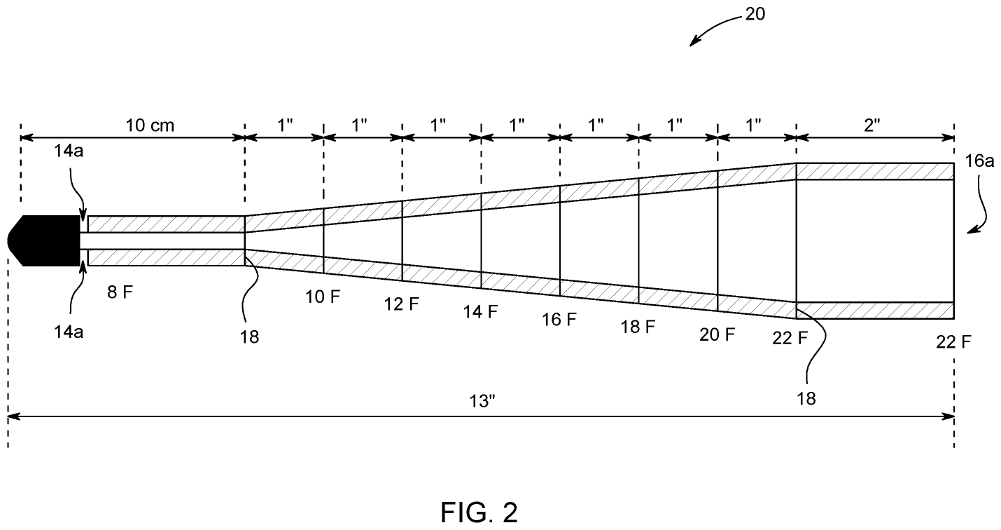

As shown in FIG. 2 illustrating schematic view of feeding tube orifice dilator with series of measuring marks and is hollow on the inside, in accordance with an embodiment of the present subject matter. The plurality of feeding tube orifice dilators (10, 20, 30, 40) comprises second feeding tube orifice dilator 20 has a series of measuring marks 18 which is increased progressively in diameter through the proximal end portion 14 to the distal end portion 16. The total length of the dilator 20 is at range of 13-15 inches.

Accordingly, the dilator 20 allows the users to reinstall the feeding tube into orifices that have contracted to diameter too small to reinstall the original tube such that the dilator 20 can also be used to replace the feeding tube with a larger diameter feeding tube if needed. As shown in FIG. 2 as Model 2 which is same as Model 1, this dilator 20 has one or two small openings near the tip (not at the tip) (14a), and is hollow on inside and ends with an open end (16a) to allow a whoosh of air flow (an indirect hint that the dilator's tip entered the stomach).

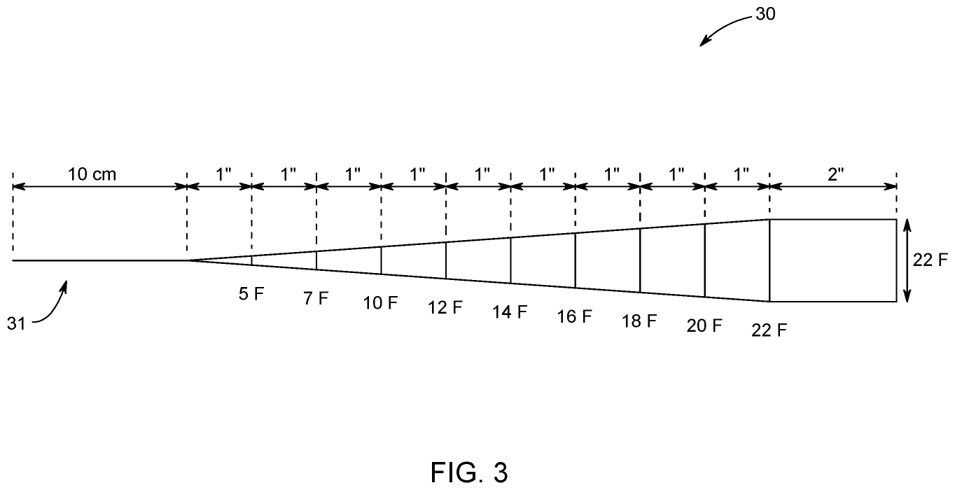

As shown in FIG. 3 illustrating schematic view of feeding tube orifice dilator with soft tipped wire, in accordance with an embodiment of the present subject matter. The plurality of feeding tube orifice dilators (10, 20, 30, 40) comprises a third feeding tube orifice dilator 30 that begins with a sharp long tip to allow insertion into a very tight orifice. Further, the dilator 30 progressively increases in dimension as shown by a series of measuring markings 18. Further, the dilator 30 that includes a soft tipped wire 31 inserted into the proximal end portion 14 to the distal end portion 16. As shown in FIG. 3 as Model 3, starts with a soft tipped wire 0.038″ in diameter×10 cm and is attached to a solid flexible but firm rubber or plastic tube that slowly dilates as per the drawing. The dilator 30 is solid on the inside.

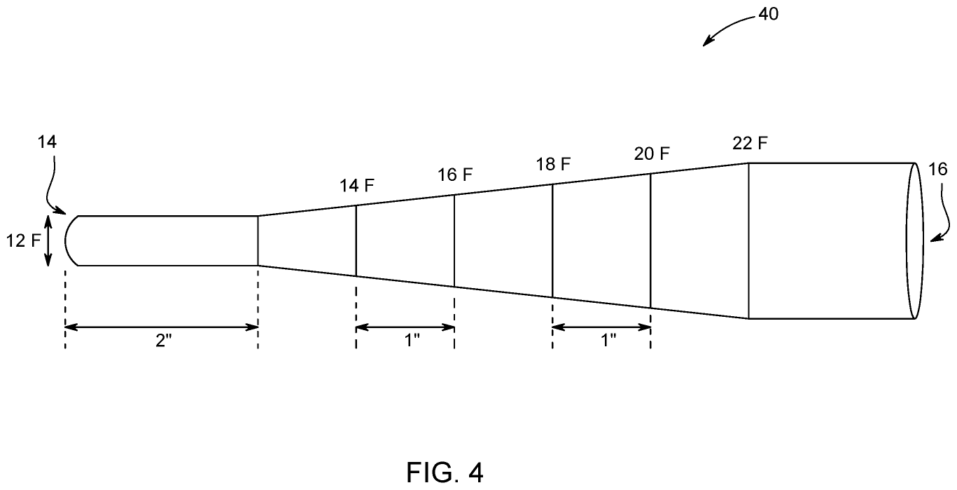

As shown in FIG. 4 illustrating schematic view of feeding tube orifice dilator with proximal and distal end portions, in accordance with an embodiment of the present subject matter. The plurality of feeding tube orifice dilators (10, 20, 30, 40) comprises a fourth feeding tube orifice dilator 40 is provided with a proximal end 14 and a distal end 16. The diameter of the proximal end portion 14 is at 12 F and diameter of the distal end portion 16 is at 22 F. As shown in FIG. 4 as Model 4 which has measurement as per the FIG. 4. It is solid since it is to be used only when inserting the dilator in not a problem and the purpose is only to make the orifice somewhat larger.

In accordance with an embodiment of the present invention relates to the dilator 40 which is inserted and removed prior to inserting the feeding tube after the opening is enlarged. Further, the dilator 40 is intended when only further dilation of the PEG orifice is needed.

Accordingly, the dilator 40 doesn't start with a sharp tip and is intended when only further dilation of the PEG orifice is needed but insertion of the dilator 40 into the PEG orifice is not a problem.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching present invention. The exemplary embodiment is chosen and described in order to best explain the principles of the present invention and its practical application, to thereby enable others skilled in the art to best utilize the present invention and various embodiments with various modifications as are suited to the particular use contemplated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances, where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It will be further appreciated that functions or structures of a plurality of components or steps may be combined into a single component or step, or the functions or structures of one-step or component may be split among plural steps or components. The present invention contemplates all of these combinations. Unless stated otherwise, dimensions and geometries of the various structures depicted herein are not intended to be restrictive of the invention, and other dimensions or geometries are possible. In addition, while a feature of the present invention may have been described in the context of only one of the illustrated embodiments, such feature may be combined with one or more other features of other embodiments, for any given application. It will also be appreciated from the above that the fabrication of the unique structures herein and the operation thereof also constitute methods in accordance with the present invention. The present invention also encompasses intermediate and end products resulting from the practice of the methods herein. The use of “comprising” or “including” also contemplates embodiments that “consist essentially of” or “consist of” the recited feature.

Although embodiments for the present subject matter have been described in language specific to structural features, it is to be understood that the present subject matter is not necessarily limited to the specific features described. Rather, the specific features and methods are disclosed as embodiments for the present subject matter. Numerous modifications and adaptations of the system/component of the present invention will be apparent to those skilled in the art, and thus it is intended by the appended claims to cover all such modifications and adaptations which fall within the scope of the present subject matter.

What is claimed is:

1. A plurality of feeding tube orifice dilators (10, 20, 30, 40) that allows users to dilate the Percutaneous endoscopic gastrostomy (PEG) orifice, comprising:
a proximal end portion (14) and a distal end portion (16) interconnected by an elongated body (12);
a series of measuring marks (18) is applied to the feeding tube orifice dilators (10, 20, 30, 40) which is increased progressively in diameter through the proximal end portion (14) to the distal end portion (16); and
a soft tipped wire (31) inserted into the proximal end portion (14) to the distal end portion (16) in dilator (30).

2. The plurality of feeding tube orifice dilators (10, 20, 30, 40) as claimed in claim 1, wherein dimensions of the dilators (10, 20, 30) are varying between 8 F and 22 F.

3. The plurality of feeding tube orifice dilators (10, 20, 30, 40) as claimed in claim 1, wherein the dilators (10, 20, 30, 40) are made of non-allergenic, flexible plastic or rubber or silicone materials.

4. The plurality of feeding tube orifice dilators (10, 20, 30, 40) as claimed in claim 1, wherein the distal end portion (16) in dilators (10, 30, 40) is substantially solid.

5. The plurality of feeding tube orifice dilators (10, 20, 30, 40) includes in dilator (20) a proximal end openings (14a) and a distal end opening (16a) and is hollow on the inside.

6. The plurality of feeding tube orifice dilators (10, 20, 30, 40) as claimed in claim 5, wherein the proximal end opening (14a) and distal end opening (16a) in dilator (20) is to hear whoosh of air flow. It could be also used to inject x-ray contrast to verify position further, if needed.

7. The plurality of feeding tube orifice dilators (10, 20, 30, 40) as claimed in claim 1, wherein diameter of the proximal end (14) of dilator (40) is at 12 F and diameter of the distal end portion (16) is at 22 F.

8. The plurality of feeding tube orifice dilators (10, 20, 30, 40) as claimed in claim 5, wherein the proximal end (14) is begun the dilator (30) with the soft tipped wire (31).

9. The plurality of feeding tube orifice dilators (10, 20, 30, 40) as claimed in claim 1, wherein the dilator (10, 20, 30, 40) is inserted and removed prior to inserting the feeding tube after the opening is enlarged.

10. The plurality of feeding tube orifice dilators (10, 20, 30, 40) as claimed in claim 1, wherein the dilator (40) is intended when only further dilation of the PEG orifice is needed.

11. The plurality of feeding tube orifice dilators (10, 20, 30, 40) as claimed in claim 1, wherein the dilator (30) the diameter of soft tipped wire (31) is at 0.038 inches, as claimed in claim 1, wherein soft tipped wire (31) is very flexible.

12. The plurality of feeding tube orifice dilators (10, 20, 30, 40) as claimed in claim 1, wherein total length of the dilators (10, 20) is at range of 13-15 inches.
